(19) United States
(12) Patent Application Publication
(10) Pub. No.: US 2015/0274621 A1
(43) Pub. Date: Oct. 1, 2015

(54) Homogeneous Hydrogenation of Esters Employing a Complex of Iron as Catalyst

(71) Applicants: The Procter & Gamble Company, Cincinnati, OH (US); University of Cincinnati, Cincinnati, OH (US)

(72) Inventors: Neil Thomas FAIRWEATHER, Liberty Township, OH (US); Michael Steven GIBSON, Cincinnati, OH (US); Hairong GUAN, Cincinnati, OH (US); Sumit CHAKRABORTY, Rochester, NY (US); Huiguang DAI, Cincinnati, OH (US); Papri BHATTACHARYA, Fort Thomas, KY (US)

(21) Appl. No.: 14/664,966

(22) Filed: Mar. 23, 2015

Related U.S. Application Data
(60) Provisional application No. 61/972,927, filed on Mar. 31, 2014.

(51) Int. Cl. C07C 29/149; B01J 31/16; B01J 31/20; B01J 31/24; B01J 31/22

(52) U.S. Cl. CPC C07C 29/149; B01J 31/2476; B01J 31/2295; B01J 31/20; B01J 31/16; B01J 2231/64; B01J 2231/643; B01J 2531/842

(57) ABSTRACT

The homogeneous hydrogenation of organic carbonyls, especially esters, under relatively mild conditions using iron hydrido-borohydride catalyst complexes having amino-phosphine pincer ligands. The catalyst and process are well-suited for catalyzing the hydrogenation of a wide variety of organic carbonyls, such as hydrogenation of fatty acid esters to alcohols. In particular embodiments, the process can be carried out in the absence of solvent.

### FIELD OF THE INVENTION

The present invention relates to a homogenous process for the hydrogenation of organic carbonyl compounds.

### BACKGROUND OF THE INVENTION

Hydrogenation of esters is an industrially important process and is used to manufacture alcohols on a multi-million ton scale per annum for numerous applications. Long-chain or fatty alcohols, in particular, are widely used as precursors to surfactants, plasticizers, and solvents. In 2012, world consumption of fatty alcohols grew to 2.2 million metric tons, and the global demand was projected to increase at a compound annual growth rate of 3-4% from 2012 to 2020. Currently, about 50% of fatty alcohols are considered “natural fatty alcohols” as they are produced through hydrogenation of fatty acid methyl esters that are derived from coconut and palm kernel oils, among other renewable materials.

Current technologies for the large scale ester hydrogenation to fatty alcohols (e.g. detergent length methyl esters, primarily C₁₂-C₁₄) typically utilize a heterogeneous catalysts such as copper-chromite and operate under extreme temperatures (250-300° C.) and pressures (2000-3000 psig of H₂ pressure). While effective, these processes are very energy and capital intensive. Alternatively, homogeneous catalysts containing precious metals such as ruthenium and osmium have been reported but often require large amounts of additives, such as an organic or inorganic bases and added solvents to obtain commercially acceptable yields.

Accordingly, it would be desirable to provide an alternative method to transform esters to alcohols under less harsh conditions (e.g., temperature, pressure), thereby leading to reduced energy and capital expenditures. It would also be desirable if the hydrogenation process is more environmentally friendly, generating no or only minimal waste, and not requiring the use of precious metals. Further, it would be advantageous to provide a method whereby refined oils can be directly converted to alcohols through hydrogenation without the need to first convert the oils to fatty acid methyl esters.

### SUMMARY OF THE INVENTION

The present invention provides a homogeneous method for the hydrogenation of esters under relatively mild conditions by employing molecular catalysts based on iron, which is an earth abundant and environmentally benign metal. The method is well-suited for catalyzing the hydrogenation of a wide variety of organic carbonyls without generating non-alcohol byproducts. The homogeneous method comprises contacting organic carbonyls with molecular hydrogen (H₂) in the presence of the iron-based catalyst. Further, the method is effective for the conversion of refined oils, such as coconut or palm, directly to detergent-length alcohols without the addition of solvent (“neat”) thus eliminating or minimizing the generation of harmful wastes.

### BRIEF DESCRIPTION OF THE DRAWINGS

FIG. 1 is a proposed catalytic cycle for the hydrogenation of esters to alcohols using the compound of Formula 2

### DETAILED DESCRIPTION OF THE INVENTION

The present invention provides a method of hydrogenating a carbonyl compound to produce a hydrogenated reaction product. The method comprises contacting the carbonyl compound with molecular hydrogen in the presence of an iron hydrido-borohydride catalyst complex having amino-phosphine pincer ligands and represented by the formula:

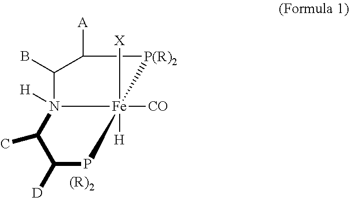

wherein each R is independently selected from aromatic moieties and alkyl moieties; X is selected from hydrogen and borohydride; and A, B, C, and D are each independently selected from hydrogen, aromatic moieties, and alkyl moieties. The method herein provides efficient, inexpensive hydrogenation of esters (e.g., aromatic, aliphatic, fatty acid esters) under mild conditions.

For example, one iteration of the iron hydrido-borohydride catalyst complex of the present invention can be represented by the formula:

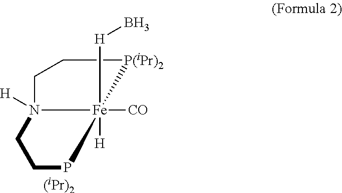

Any suitable carbonyl compounds, such as esters, amides, aldehydes, and ketones, can be hydrogenated using the present method. For example, such esters can include aromatic, aliphatic, methyl, isopropyl, butyl, long-chained, branched, non-branched, primary, secondary, wax ester, and glyceride. In certain aspects, the carbonyl compound can be a fatty acid ester. The fatty acid ester chain can typically have from 3 to 40, or from 10 to 20, carbon atoms.

Typically, the step of contacting the carbonyl compound with molecular hydrogen is performed at a temperature of from 20° C. to 200° C. and a pressure of from 50 to 2000 psig, or from 500 to 1200 psig, or from 700 to 800 psig. The carbonyl compound is part of a reaction mixture that comprises, consists of, or consists essentially of the carbonyl compound. The catalyst is included in an effective amount to facilitate the reaction. For example, catalyst can be present at a level of from 0.02 to 5 mole %, or from 0.02 to 10 mole %, or from 0.5 to 2.0 mole %. Using this method, the hydrogenated reaction product yield range from 5% to 100%, from 25% to 99%, or from 60% to 99% in particular iterations.

In certain aspects, the method does not comprise the addition of exogenous solvent. As used herein, “exogenous solvent” means solvent added to the reaction mixture above the amount that may already be inherently present in the reaction mixture. For example, exogenous solvent would include solvent added as a reaction dilution solvent, such as toluene, tetrahydrofuran (THF), dioxane, methanol, ethanol, and combinations thereof.

In another aspect, the invention provides a method of reducing an ester moiety to an alcohol moiety. The method comprises contacting the ester moiety with a catalyst represented by Formula 1, as above.

In some iterations, A and B collectively are members of a first cyclic moiety that can be either aromatic or alkyl, and that has five or six members; and where C and D collectively are members of a second cyclic moiety that can be either aromatic or alkyl, and that has five or six members. In others, each of A, B, C, and D are a hydrogen atom.

In some iterations of the method of reducing an ester moiety to an alcohol moiety, the catalyst has the formula represented by Formula 2, above. In yet another aspect, the method of reducing an ester moiety to an alcohol moiety comprises contacting the ester moiety with a catalyst complex represented by the formula:

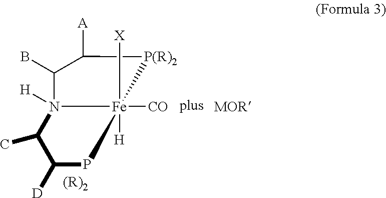

wherein each R is independently selected from aromatic moieties and alkyl moieties; X is selected from borohydride, chloride, bromide, and iodide; A, B, C, and D are each independently selected from hydrogen, aromatic moieties, and alkyl moieties; and MOR′ represents sodium methoxide or potassium tertiary butoxide.

In some cases, A and B collectively are members of a first cyclic moiety that is aromatic or alkyl, and that has five or six members; and where C and D collectively are members of a second cyclic moiety that is aromatic or alkyl, and that has five or six members. In others, each of A, B, C, and D are hydrogen atoms.

In additional aspects, the catalyst complex for reducing ester to alcohol is represented by the formula:

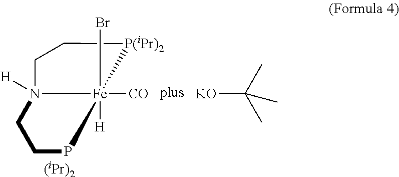

Synthesis of the iron pincer hydrido borohydride complex herein can be accomplished in two steps, as shown by Equations I and II below.

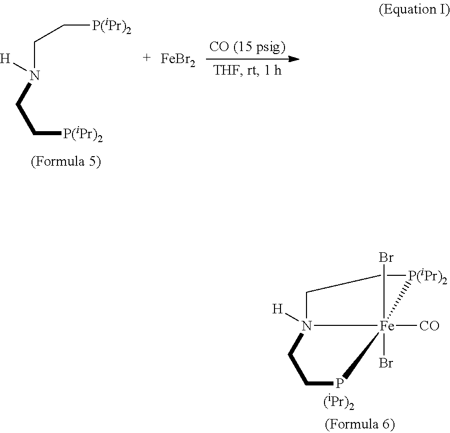

In the first step, the ^{iPr}PN(H)P pincer ligand (Formula 5) is treated with anhydrous FeBr₂ and CO (15 psig) in THF that results in a deep blue iron pincer hydrido borohydride complex using the following procedure. Example 1A exemplifies this synthesis step.

The desired complex (Formula 2) is prepared from that of Formula 6 in 85% yields by a reaction with an excess of NaBH₄, as shown by Equation II. Example 1B herein exemplifies this synthesis step.

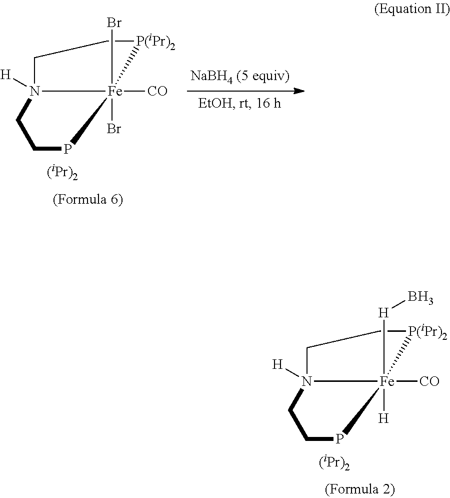

An iron monohydride complex (Formula 7) can also be synthesized similarly from Formula 6 employing one equivalent of NaBH₄ (Equation 3). Example 1C herein exemplifies this synthesis step.

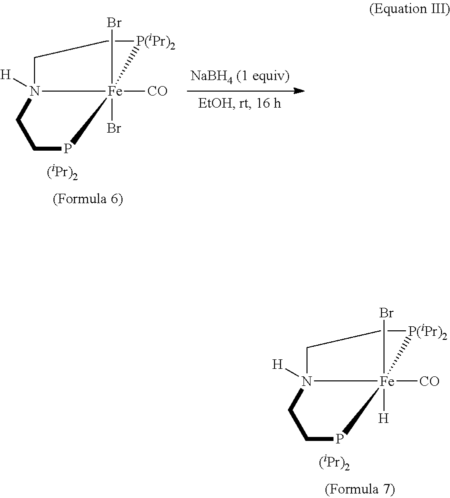

This catalytic system is also effective for the conversion of coconut oil derived fatty acid methyl esters to detergent alcohols without adding exogenous solvent (performed “neat”).

### EXAMPLES

### Example 1

### Catalyst Synthesis

### Example 1A

### Synthesis of [^{iPr}PN(H)P]Fe(CO)Br₂ (Formula 6)

In a glovebox, a 100 mL oven-dried Schlenk flask equipped with a stir bar was charged with anhydrous FeBr₂ (510 mg, 2.36 mmol) and 30 mL of THF, which resulted in an orange solution. A THF solution of (ⁱPr₂PCH₂CH₂)NH (10 wt %, 9.0 mL, 2.60 mmol) was added and, upon mixing with the FeBr₂ solution for a few minutes, a thick white precipitate formed. The flask was connected to a Schlenk line, and the argon inside the flask was replaced with CO by performing a freeze-pump-thaw cycle. When mixed with CO and warmed to room temperature, the white precipitate quickly dissolved to yield a deep blue solution. The solution was stirred under 15 psig of CO for 1 h followed by evaporation to dryness under vacuum. The resulting blue residue was washed with pentane (15 mL×3) and dried under vacuum to give the titled compound as a blue powder (1.20 g, 93% yield). The ¹H NMR spectra of this complex showed broad resonances, presumably due to a small amount of paramagnetic impurity. This compound can be exposed to air briefly without significant decomposition. ¹H NMR (400 MHz, CD₂Cl₂, δ): 1.42 (br, PCH(CH₃)₂, 24H), 2.09 (br, CH₂, 2H), 2.51 (br, CH₂, 2H), 2.77 (br, PCH(CH₃)₂, 4H), 3.46 (br, CH₂, 2H), 3.69 (br, CH₂, 2H), 5.39 (br, NH, 1H). ¹H NMR (400 MHz, C₆D₆, δ): 1.22-1.26 (m, PCH(CH₃)₂, 12H), 1.30-1.48 (m, PCH(CH₃)₂, 12H), 1.52-1.68 (m, CH₂, 2H), 1.80-1.92 (m, CH₂, 2H), 2.70-2.88 (m, PCH(CH₃)₂+CH₂, 6H), 3.13-3.24 (m, CH₂, 2H), 4.87 (t, ³J_{P-H}=12 Hz, NH, 1H). ¹³C{¹H} NMR (101 MHz, CD₂Cl₂, δ): 19.16 (s, PCH(CH₃)₂)_{,} 19.47 (s, PCH(CH₃)₂)_{,} 19.93 (s, PCH(CH₃)₂), 20.38 (s, PCH(CH₃)₂)_{,} 23.81 (t, J_{C-P}=9.6 Hz, PCH(CH₃)₂), 25.49 (t, J_{C-P}=11.1 Hz, PCH(CH₃)₂), 26.94 (t, J_{C-P}=6.7 Hz, NCH₂CH₂), 50.80 (t, J_{C-P}=4.3 Hz, NCH₂CH₂), 227.29 (t, J_{C-P}=22.4 Hz, FeCO). ³¹P{¹H} NMR (162 MHz, CD₂Cl₂, δ): 68.4 (s). ³¹P{¹H} NMR (162 MHz, C₆D₆, δ): 68.4 (s). ATR-IR (solid): ν(N—H)=3188 cm⁻¹, ν(CO)=1951 and 1928 cm⁻¹. Transmission-IR (in THF): ν(CO)=1941 cm⁻¹. Anal. Calcd for C₁₇H₃₇NOP₂Br₂Fe: C, 37.19; H, 6.79; N, 2.55; Br, 29.10. Found: C, 37.36; H, 6.77; N, 2.63; Br, 29.22.

### Example 1B

### Synthesis of [^{iPr}PN(H)P]Fe(H)(CO)(BH₄) (Formula 2)

Under an argon atmosphere, a 100 mL oven-dried Schlenk flask equipped with a stir bar was charged with Formula 6 (400 mg, 0.73 mmol) and NaBH₄ (138 mg, 3.65 mmol). Adding 50 mL of dry and degassed ethanol to this mixture at 0° C. at first resulted in a green solution, which changed its color to yellow within a few minutes. The resulting mixture was gradually warmed to room temperature and then stirred for additional 16 h. Removal of the volatiles under vacuum afforded a yellow solid, which was treated with 80 mL of toluene and then filtered through a pad of Celite to give a yellow solution. Evaporating the solvent under vacuum yielded the desired compound as a bright yellow powder (250 mg, 85% yield). This compound can be exposed to air briefly without significant decomposition.

[^{iPr}PN(H)P]Fe(D)(CO)(BD₄) (Formula 2-d₅) were synthesized similarly from Formula 6 and NaBD₄. ¹H NMR (400 MHz, C₆D₆, δ): −19.52 (t, J_{P-H}=50.4 Hz, FeH, 1H), −2.73 (br, FeBH₄, 4H), 0.86-0.91 (m, PCH(CH₃)₂, 6H), 1.08-1.11 (m, PCH(CH₃)₂, 6H), 1.16-1.21 (m, PCH(CH₃)₂, 6H), 1.47-1.60 (m, PCH(CH₃)₂+PCH(CH₃)₂, 10H), 1.67-1.71 (m, CH₂, 2H), 1.97-2.01 (m, CH₂, 2H), 2.36-2.40 (m, CH₂, 2H), 2.76-2.79 (m, CH₂, 2H), 3.87 (br, NH, 1H). ¹³C{¹H} NMR (101 MHz, C₆D₆, δ): 18.42 (s, PCH(CH₃)₂)_{,} 19.17 (s, PCH(CH₃)₂)_{,} 20.58 (s, PCH(CH₃)₂), 20.94 (s, PCH(CH₃)₂)_{,} 25.40 (t, J_{C-P}=12.8 Hz, PCH(CH₃)₂), 29.08 (t, J_{C-P}=7.5 Hz, NCH₂CH₂), 29.74 (t, J_{C-P}=9.7 Hz, PCH(CH₃)₂), 54.17 (t, J_{C-P}=5.8 Hz, NCH₂CH₂), 222.56 (t, J_{C-P}=25.8 Hz, FeCO). ³¹P{¹H} NMR (162 MHz, C₆D₆, δ): 99.2 (s). ¹¹B NMR (128 MHz, C₆D₆, δ): −33.9 (quin, ¹J_{B-H}=77.9 Hz). ¹¹B{¹H} NMR (128 MHz, C₆D₆, δ): −33.9 (s). ATR-IR of Formula 2 (solid): ν(N—H)=3197 cm⁻¹, ν(B—Hₜₑᵣₘᵢₙₐₗ)=2357 cm⁻¹, ν(B—H_{bridging})=2038 cm⁻¹, ν(CO)=1896 cm⁻¹, ν(FeH)=1832 cm⁻¹. ATR-IR of Formula 2-d₅ (solid): ν(N—H)=3198 cm⁻¹, ν(B-Dₜₑᵣₘᵢₙₐₗ)=1772 cm⁻¹, ν(B-D_{bridging})=1493 cm⁻¹, ν(CO)=1895 cm⁻¹, ν(FeD)=1327 cm⁻¹. Anal. Calcd. for C₁₇H₄₂BNOP₂Fe: C, 50.40; H, 10.45; N, 3.46. Found: C, 50.34; H, 10.25; N, 3.36.

### Example 1C

### Synthesis of [^{iPr}PN(H)P]Fe(H)(CO)(Br) (Formula 7)

Under an argon atmosphere, a 100 mL oven-dried Schlenk flask equipped with a stir bar was charged with Formula 6 (100 mg, 0.182 mmol) and NaBH₄ (7.0 mg, 0.185 mmol). Adding 15 mL of dry and degassed ethanol to this mixture at 0° C. at first resulted in a green solution, which changed its color to orange within a few minutes. The resulting mixture was gradually warmed to room temperature and then stirred for additional 16 h. Removal of the volatiles under vacuum afforded an orange solid, which was treated with 40 mL of toluene and then filtered through a pad of Celite to give an orange solution. After the solution was concentrated to ˜3 mL under vacuum, it was carefully layered with ˜10 mL of pentane and placed in a refrigerator (0° C.). Orange crystals of the desired compound formed within a day. Decantation of the top layer using a cannula followed by solvent evaporation afforded the titled compound (60 mg, 70% yield). This compound is air sensitive and should be handled under an inert atmosphere. ¹H NMR (400 MHz, C₆D₆, δ): −22.77 (t, J_{P-H}=52.0 Hz, FeH, 1H), 0.86 (br, PCH(CH₃)₂, 6H), 1.12 (br, PCH(CH₃)₂, 6H), 1.22 (br, PCH(CH₃)₂, 6H), 1.58-1.69 (m, CH₂+PCH(CH₃)₂+PCH(CH₃)₂, 12H), 2.03 (br, CH₂, 2H), 2.64 (br, CH₂, 2H), 3.07 (br, CH₂, 2H), 3.55 (br, NH, 1H). ¹H NMR (400 MHz, THF-d₈, δ): −22.63 (t, ³J_{P-H}=52.0 Hz, FeH, 1H), 1.07-1.12 (m, PCH(CH₃)₂, 6H), 1.19-1.25 (m, PCH(CH₃)₂, 6H), 1.29-1.33 (m, PCH(CH₃)₂, 6H), 1.48-1.54 (m, PCH(CH₃)₂, 6H), 1.70-1.82 (m, PCH(CH₃)₂, 2H), 2.08-2.18 (m, PCH(CH₃)₂, 2H), 2.22-2.34 (m, CH₂, 2H), 2.35-2.44 (m, CH₂, 2H), 2.81-2.95 (m, CH₂, 2H), 3.18-3.34 (m, CH₂, 2H), 3.59-3.72 (m, NH, 1H). ¹³C{¹H}NMR (101 MHz, C₆D₆, δ): 18.08 (s, PCH(CH₃)₂)_{,} 19.19 (s, PCH(CH₃)₂)_{,} 20.70 (s, PCH(CH₃)₂), 20.86 (s, PCH(CH₃)₂), 24.70 (t, J_{C-P}=12.1 Hz, PCH(CH₃)₂), 28.45 (t, J_{C-P}=10.1 Hz, PCH(CH₃)₂), 29.63 (t, J_{C-P}=8.1 Hz, NCH₂CH₂), 53.72 (t, J_{C-P}=6.1 Hz, NCH₂CH₂), 224.18 (t, J_{C-P}=26.3 Hz, FeCO). ³¹P{¹H} NMR (162 MHz, C₆D₆, δ): 93.5 (d, J_{P-H}=9.7 Hz, residual coupling due to incomplete decoupling of the high-field hydride resonance). ATR-IR (solid): ν(N—H)=3173 cm⁻¹, ν(CO)=1894 cm⁻¹, ν(FeH)=1852 cm⁻¹. Anal. Calcd for C₁₇H₃₈NOP₂BrFe: C, 43.43; H, 8.15; N, 2.98; Br, 16.99. Found: C, 43.47; H, 8.20; N, 2.93; Br, 16.77.

### Example 2

### Optimization of the Catalytic Conditions

In a glovebox, an iron complex (Formula 2, 6, or 7; 25 μmol), additive (if needed), methyl benzoate (105 μL, 833 μmol), and tridecane (80 μL, 328 μmol, internal standard) were mixed with 0.5 mL of solvent in a small test tube, which was placed in a HEL CAT18 high-pressure vessel. The vessel was sealed, flushed with H₂ three times, and placed under an appropriate H₂ pressure. The vessel was then heated by an oil bath at appropriate temperature. A small aliquot was withdrawn from the test tube and diluted with approximately 4 mL of ethyl acetate prior to GC analysis. The percentage conversion for each reaction was calculated by comparing the integration of methyl benzoate with that of the internal standard. The results are summarized in Table 1 below.

TABLE 1
Catalytic activity of iron complexes for the hydrogenation of methyl benzoate.

| | | | | | | PhCH₂OH |
|---|---|---|---|---|---|---|
| Catalyst | Pressure | Temp. | Time | Solvent | Conversion | Yield |
| Formula 6 | 150 psig | 115° C. | 3 h | THF | 0% | 0% |
| (3 mol %)/NaBH₄ (15 mol %) | | | | | | |
| Formula 6 | 150 psig | 115° C. | 3 h | THF | 0% | 0% |
| (3 mol %)/KO^{t}Bu (10 mol %) | | | | | | |
| Formula 7 | 150 psig | 115° C. | 3 h | THF | 0% | 0% |
| (3 mol %) | | | | | | |
| Formula 7 | 150 psig | 115° C. | 3 h | THF | >95% | 72% |
| (3 mol %)/KO^{t}Bu (10 mol %) | | | | | | |
| Formula 2 (3 mol %) | 150 psig | 115° C. | 3 h | THF | 100% | 94% |
| Formula 2 (3 mol %) | 150 psig | 115° C. | 3 h | 1,4- | 100% | 92% |
| | | | | dioxane | | |
| Formula 2 (3 mol %) | 150 psig | 115° C. | 3 h | toluene | 100% | >99% |
| Formula 2 (2 mol %) | 150 psig | 115° C. | 3 h | toluene | 100% | 82% |
| Formula 2 (3 mol %) | 100 psig | 115° C. | 3 h | toluene | 82% | 44% |
| Formula 2 (3 mol %) | 60 psig | 115° C. | 3 h | toluene | 0% | 0% |
| Formula 2 (3 mol %) | 150 psig | 85° C. | 3 h | toluene | 100% | 95% |
| Formula 2 (3 mol %) | 150 psig | 60° C. | 3 h | toluene | 0% | 0% |

Formula 2 can be directly employed as a catalyst (no base is needed) for ester hydrogenation. A general scheme for this hydrogenation reaction is shown by Equation IV:

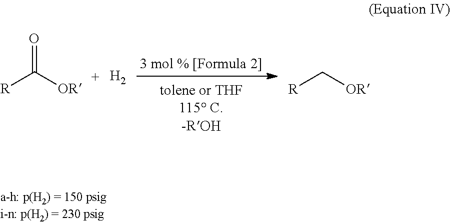

Table 2 illustrates the scope of esters that can be hydrogenated using the complex of Formula 2 as the catalyst under the aforementioned conditions.

TABLE 2
Scope of esters

| Ester | Chemical Formula | Time | Yield |
|---|---|---|---|
| a | 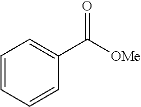 | 3 h | 92% |
| b | 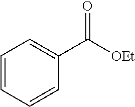 [SMILES: CCOC(=O)c1ccccc1] | 3 h | 90% |
| c | 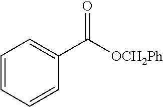 | 3 h | 95% |
| d | 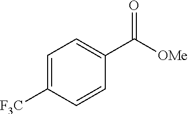 [SMILES: COC(=O)c1ccc(C(F)(F)F)cc1] | 1.5 h | 94% |
| e | 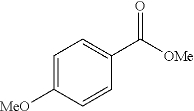 [SMILES: COC(=O)c1ccc(OC)cc1] | 12 h | 96% |
| f | 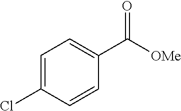 [SMILES: COC(=O)c1ccc(Cl)cc1] | 3 h | 88% |
| g | 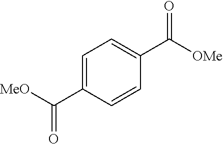 [SMILES: COC(=O)c1ccc(C(=O)OC)cc1] | 24 h | 63% |
| h | 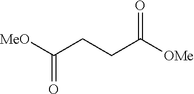 | 24 h | 75% |
| i | 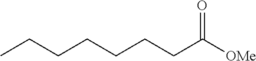 | 24 h | 72% |
| j | 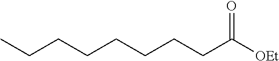 | 24 h | 91% |
| k | 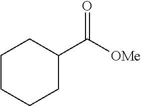 | 24 h | 50% |
| l | 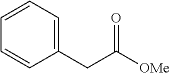 [SMILES: COC(=O)Cc1ccccc1] | 24 h | 85% |
| m | 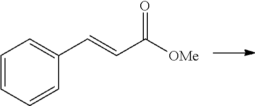 [SMILES: COC(=O)/C=C/c1ccccc1] | 24 h | 93% |
| | 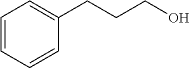 | | |
| n | 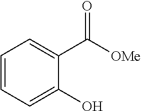 [SMILES: COC(=O)c1ccccc1O] | 24 h | 0% |

Unsubstituted aromatic esters such as methyl benzoate, ethyl benzoate, and benzyl benzoate were hydrogenated to the benzyl alcohol with high isolated yields (90-95%). Aromatic methyl esters containing —CF₃, —OMe, and —Cl substituents at the para position reacted smoothly under these conditions to afford the corresponding alcohols in good yields. Esters containing electron-withdrawing groups (—CF₃, —Cl) reacted faster than the one with electron-donating substituent (—OMe). More challenging aromatic and aliphatic diester substrates were also hydrogenated successfully, albeit with slower catalytic turnovers.

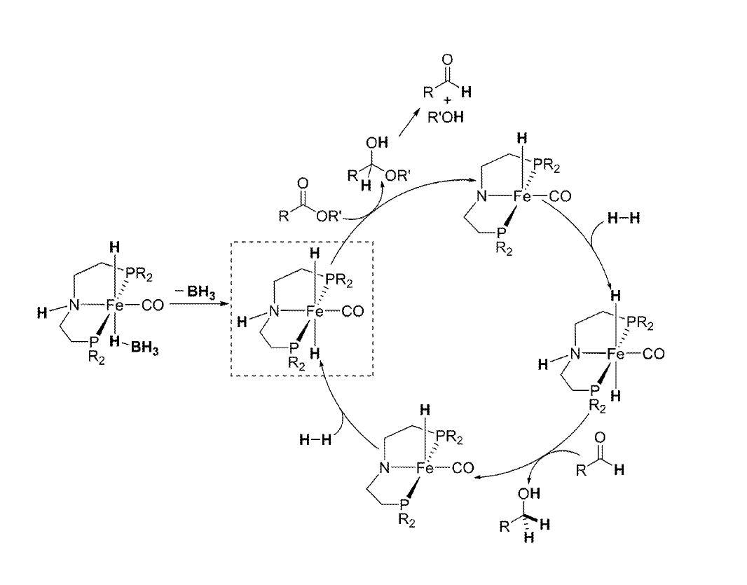

It is believed that under the catalytic conditions, BH₃ dissociates from the complex of Formula 2 to release the active trans-dihydride species. The acidic NH and the hydridic FeH hydrogens can now be transferred simultaneously to the ester substrate to yield a hemiacetal intermediate and a 5-coordinate iron species, which is converted back to the trans-dihydride via the uptake of H₂. The hemiacetal intermediate can dissociate into an alcohol and an aldehyde, which is further reduced by the trans-dihydride. The proposed catalytic cycle for the hydrogenation of esters to alcohols using the compound of Formula 2 is shown in FIG. 1.

### Example 3

### Neat Hydrogenation of Fatty Acid Methyl Esters

### Example 3A

### Small Scale (22 mL Parr reactor)

Methyl ester (Procter & Gamble Chemicals CE-1270) and catalyst (˜1 mole %) were added to a 22 mL Parr reactor along with a magnetic stir bar. The reactor was closed, flushed with H₂, pressurized and placed in a pre-heated aluminum heating block (135° C.). After the determined period of time, the reactor was cooled, the pressure vented, opened and a sample removed for analysis by GC to determine the yield of alcohol formation. Selected results are in Table 3 below.

These are believed to be the first successful hydrogenation of esters carried out under neat conditions using a homogeneous Fe-based catalyst.

TABLE 3

| Catalyst | Pressure (psig) | Time (h) | % Yield Alcohol |
|---|---|---|---|
| Formula 2 | 750 | 3 | 98.6 |
| Formula 2 | 300 | 3 | 72.6 |
| Formula 2 | 750 | 3 | 98.6 |
| Formula 2 | 750 | 1 | 96.2 |
| Formula 2 | 750 | 3 | 98.5 |

### Example 3B

### Larger Scale (300 mL Parr reactor)

To a 300 mL high pressure stainless steel Parr reactor were added iron catalyst (Formula 2, 0.72 g, 0.26 mol %), and CE-1270 (149.96 g, 676.2 mmol). The reactor was sealed, flushed with H₂ (4×) followed by pressuring to 750 psig. Stirring was started (˜1000 rpm) and the reactor set to warm to 135° C. Time=0 was started when the reaction had reached 135° C. The reaction was continued under these conditions for 3 hours with samples removed for GC analysis at time=0 minutes, 20 minutes, 40 minutes, 1 hour, 2 hours and 3 hours. For each sample, the conversion, selectivity and alcohol yield were determined with results shown in the Table 4.

TABLE 4

| Time | | % Conversion | % Selectivity | % Yield |
|---|---|---|---|---|
| 0 | minutes | 2.3 | 100.0 | 2.3 |
| 20 | minutes | 24.5 | 95.7 | 23.4 |
| 40 | minutes | 26.2 | 93.7 | 24.6 |
| 1 | hour | 26.7 | 93.0 | 24.8 |
| 2 | hours | 27.5 | 90.9 | 24.9 |
| 3 | hours | 28.1 | 88.8 | 25.0 |

### Example 3C

### Lower Temperature (300 mL Parr reactor)

To a 300 mL high pressure stainless steel Parr reactor were added iron catalyst (Formula 2, 0.74 g, 0.27 mol %), and CE-1270 (149.96 g, 676.2 mmol). The reactor was sealed, flushed with H₂ (4×) followed by pressuring to 750 psig. Stirring was started (˜1000 rpm) and the reactor set to warm to 115° C. Time=0 was started when the reaction had reached 115° C. The reaction was continued under these conditions for 3 hours with samples removed for GC analysis at time=0 minutes, 20 minutes, 40 minutes, 1 hour, 2 hours and 3 hours. For each sample, the conversion, selectivity and alcohol yield were determined with results shown in Table 5.

TABLE 5

| Time | | % Conversion | % Selectivity | % Yield |
|---|---|---|---|---|
| 0 | minutes | 0.0 | 0 | 0.0 |
| 20 | minutes | 19.4 | 97.0 | 18.8 |
| 40 | minutes | 34.1 | 93.7 | 32.0 |
| 1 | hour | 40.0 | 92.2 | 36.9 |
| 2 | hours | 44.3 | 90.0 | 39.8 |
| 3 | hours | 45.4 | 88.6 | 40.2 |

### Example 4

### Neat Hydrogenation of Oil Directly to Fatty Alcohols

Refined, bleached and deodorized Coconut oil (Procter & Gamble Chemicals) and catalyst (˜2 weight %) were added to a 22 mL Parr reactor along with a magnetic stir bar. The reactor was closed, flushed with H₂, pressurized and placed in a pre-heated aluminum heating block (135° C.). After stirring for 23 hours, the reactor was cooled, the pressure vented, opened and a sample removed for analysis by GC to determine the yield of alcohol formation. 11.67% fatty alcohol (C₈-C₁₆) was obtained. The C₁₈ alcohol was not tabulated as it was not able to be clearly discerned from other peaks in that range on the GC chromatogram.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A homogeneous method of hydrogenating a carbonyl compound to produce a hydrogenated reaction product, comprising contacting said carbonyl compound with molecular hydrogen in the presence of an iron hydrido-borohydride catalyst complex having amino-phosphine pincer ligands and represented by the formula: 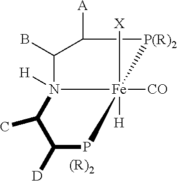
wherein each R is independently selected from aromatic moieties and alkyl moieties; X is selected from hydrogen and borohydride; and A, B, C, and D are each independently selected from hydrogen, aromatic moieties, and alkyl moieties.

2. The method of claim 1, wherein said carbonyl compound is an ester.

3. The method of claim 2, wherein said ester is selected from the group consisting of aromatic, aliphatic, methyl, isopropyl, butyl, long-chained, branched, non-branched, primary, secondary, wax ester, and glyceride.

4. The method of claim 2, wherein said carbonyl compound is a fatty acid ester.

5. The method of claim 4, wherein said fatty acid ester has from 3 to 40 carbon atoms.

6. The method of claim 5, wherein said hydrogenated reaction product is a fatty alcohol.

7. The method of claim 1, wherein contacting the carbonyl compound with molecular hydrogen is performed at a temperature of from 20° C. to 200° C. and a pressure of from 50 to 2000 psig.

8. The method of claim 7, wherein said catalyst is present at a level of from 0.02 to 5 mole %.

9. The method of claim 8, wherein the yield of hydrogenated reaction product is from 5% to 100%.

10. The method of claim 9, not comprising the addition of exogenous solvent.

11. The method of claim 10, wherein said exogenous solvent is a reaction dilution solvent.

12. The method of claim 11, wherein said reaction dilution solvent is selected from the group consisting of toluene, tetrahydrofuran (THF), dioxane, methanol, ethanol, and combinations thereof.

13. A method of reducing an ester moiety to an alcohol moiety comprising contacting the ester moiety with a catalyst represented by the formula: 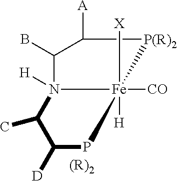
wherein each R is independently selected from aromatic moieties and alkyl moieties; X is selected from hydrogen and borohydride; and A, B, C, and D are each independently selected from hydrogen, aromatic moieties, and alkyl moieties.

14. The method of claim 13, where A and B collectively are members of a first cyclic moiety, said first cyclic moiety being aromatic or alkyl and having five or six members; and where C and D collectively are members of a second cyclic moiety, said second cyclic moiety being aromatic or alkyl and having five or six members.

15. The method of claim 13, where each of A, B, C, and D are hydrogen.

16. The method of claim 13, where the catalyst has the following formula:

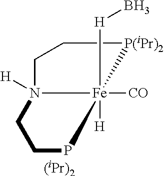

17. A method of reducing an ester moiety to an alcohol moiety comprising contacting the ester moiety with a catalyst complex represented by the formula: 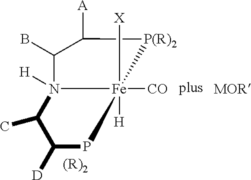
wherein each R is independently selected from aromatic moieties and alkyl moieties; X is selected from borohydride, chloride, bromide, and iodide; A, B, C, and D are each independently selected from hydrogen, aromatic moieties, and alkyl moieties; and MOR′ represents sodium methoxide, sodium ethoxide, or potassium tertiary butoxide.

18. The method of claim 17, where A and B collectively are members of a first cyclic moiety, said first cyclic moiety being aromatic or alkyl and having five or six members; and where C and D collectively are members of a second cyclic moiety, said second cyclic moiety being aromatic or alkyl and having five or six members.

19. The method of claim 17, where each of A, B, C, and D are hydrogen.

20. The method of claim 17, wherein the catalyst complex is represented by the formula: [chemical structure]
